(19) United States
(12) Patent Application Publication
(10) Pub. No.: US 2021/0236163 A1
(43) Pub. Date: Aug. 5, 2021

(54) FLEXIBLE CANNULA

(71) Applicants: Smith & Nephew, Inc., Memphis, TN (US); Smith & Nephew Orthopaedics AG, ZUG (CH); Smith & Nephew Asia Pacific Pte. Limited, Singapore (SG)

(72) Inventors: Marc J. Balboa, Hopkinton, MA (US); Geoffrey I. Kasasic, Milton, MA (US)

(21) Appl. No.: 17/048,749

(22) PCT Filed: Apr. 17, 2019
(86) PCT No.: PCT/US19/27855

§ 371 Date: Oct. 19, 2020

Related U.S. Application Data
(60) Provisional application No. 62/659,343, filed on Apr. 18, 2018.

(51) Int. Cl. A61B 17/34; A61B 17/06

(52) U.S. Cl. CPC A61B 17/3423; A61B 17/06061; A61B 2017/3484; A61B 2017/3429; A61B 2017/3443; A61B 17/3462

(57) ABSTRACT

A flexible cannula and radially extending conformable flanges used to provide an adjustable effective length of flexible cannula, adjustable for varying tissue depths. At least some of the example embodiments include a flexible tubular body having a length, a flexible distal flange, a flexible proximal flange, and one or more flexible intermediate flanges positioned on the length of the tubular body between the distal and the proximal flanges. All of the flanges may be have the equivalent outer diameters. Each flange may be spaced axially spaced apart from the adjacent flexible flange with varying spacing. Some flanges may also have a circumferential row of perforations, allowing for selective removal of some flanges. The tubular body may also have a ring of perforations, allowing for selective removal of a portion of the tubular body. The flexible cannula may also have at least one suture docking stations on a proximal end of the cannula, to selectively retain a length of suture therein.

### CROSS-REFERENCE TO RELATED APPLICATIONS

This application is a U.S. National Phase Entry of PCT Application Serial No. PCT/US19/27855 filed Apr. 17, 2019 and titled “FLEXIBLE CANNULA.” The PCT application claims the benefits of U.S. Provisional Patent Application Ser. No. 62/659,343 filed Apr. 18, 2018 titled “FLEXIBLE CANNULA”. The contents of which are incorporated by reference herein as if reproduced in full below.

### BACKGROUND

Arthroscopic surgical procedures are performed on a joint, such as a knee or shoulder, of a patient. In order to access a space within the joint, a cannula may be inserted through the tissue to provide an easy conduit for surgical instrumentation. Cannulas may be rigid tubes, which can cause damage to the tissue, or if spaced too closely together may restrict access to certain anatomies. Flexible cannulas available come in multiple lengths, so as better fit a tissue thickness or depth, which the clinician needs to determine before choosing a cannula length. In addition tissue thicknesses or depths may alter during the surgical procedure, due to patient swelling and extravasation, potentially requiring multiple cannula lengths. In addition during the procedure, suture management through the cannula can be difficult and time consuming. To more easily accommodate differing and changing tissue depths, a flexible cannula with a plurality of flexible flanges is disclosed. These flanges may be selectively removable and the cannula may include means for suture retention and management.

### BRIEF DESCRIPTION OF THE DRAWINGS

For a detailed description of example embodiments, reference will now be made to the accompanying drawings in which:

FIGS. 1A, 1B and 1C show a flexible cannula in accordance with at least some embodiments;

FIGS. 2A and 2B show a flexible cannula with perforations, in accordance with at least some embodiments;

FIGS. 3A and 3B are cross-section representations of a flexible cannula inserted into tissues of differing depths, in accordance with at least some embodiments;

FIGS. 4A and 4B show a cannula with an adjustable pawl in accordance with at least some embodiments;

FIGS. 5A, 5B and 5C show a flexible cannula with suture management slits in accordance with at least some embodiments;

FIG. 6 shows a method in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various embodiments are directed to a flexible cannula for use in procedures, such as arthroscopic or endoscopic procedures. More particularly, example embodiments are directed to flexible cannulas that comprise a plurality of flexible or conformable radial flanges along the length of the cannula, to accommodate various tissue depths, providing a ‘one-size-fits-all’ approach. The cannula has a distal flange, a proximal flange and one or more intermediate flanges positioned between the distal and proximal flanges. In use, the cannula is placed in the tissue through an incision such that the distal flange flexes open beneath the tissue as it is placed in position. Depending on the tissue depth, one of the intermediate flanges (or the proximal flange) can abut approximate to the outer surface of the tissue, keeping the cannula in place. Any length of the cannula remaining outside the tissue can be left remaining, or cut off and discarded. As tissue thickness can change, and oftentimes expand during procedures, due to general swelling and/or extravasation, some of the cannula and a corresponding flange may preferably be left attached, for later use. If any intermediate flanges are present between the joint cavity and the tissue outer surface and therefore lie along the thickness, they may be configured so as to simply flex out of the way and provide additional stability to the cannula. Alternatively, the intermediate flanges may be perforated for easy removal. The body of the cannula itself may also have multiple perforations along the body to allow for shortening the body length. The cannula can have ratchet like features and a corresponding washer with pawls to allow for an adjustable flange. The interior of the cannula can have a membrane with a slit or opening to seal fluid when instruments are placed through the cannula. The cannula may be assembled onto an obturator to facilitate insertion through the incision in the tissue.

Various embodiments are directed to methods of using a flexible cannula. The specification now turns to an example system.

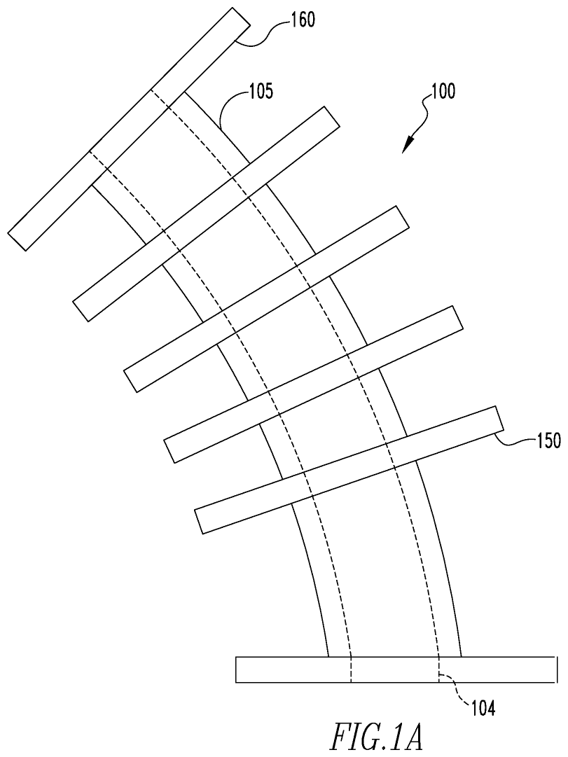

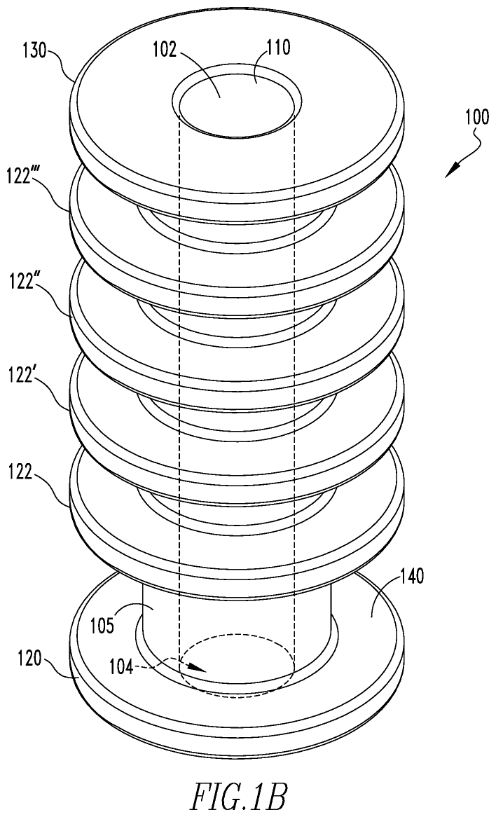

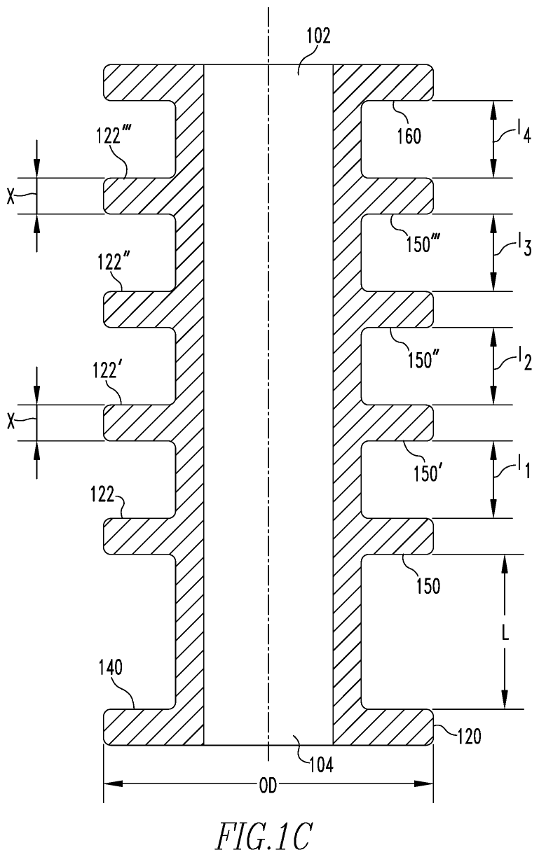

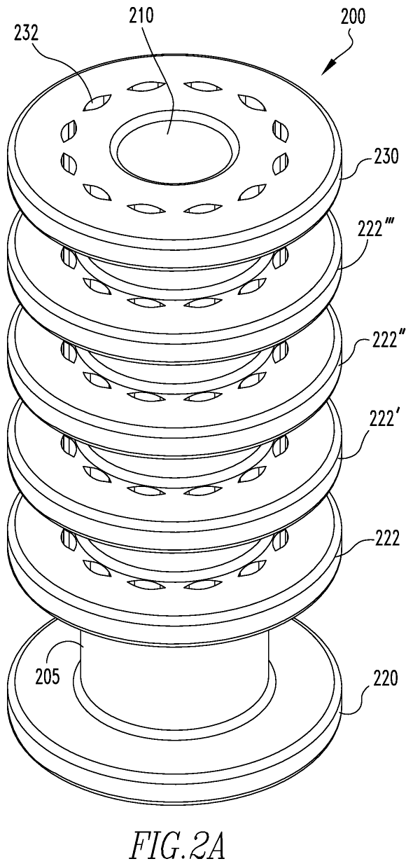

The present invention provides a surgical port or flexible cannula 100 which comprises a flexible grommet-like structure as shown in FIGS. 1A and 1B. The cannula 100 generally comprises a hollow, thin-walled flexible tubular body 105 provided with a series of thin-walled flexible annular flanges along the tubular body 105. The tubular body 105 defines a lumen 110 which includes at least one valve or slit (not shown) so as to seal fluid within the cavity as instruments are inserted through the cannula lumen 110. Both the tubular body 105 and the flanges (120, 122 122′, 122″, 122′″ and 130) consist of a flexible, resilient material, e.g., elastomeric material. A total of five flexible flanges are shown; this number varying depending on the procedure and size of patent, so that inventors envision a range of between 3 and 10 total flanges. Preferably, the tubular body 105 and the flanges have a circular cross section, although other alternative configurations, such as ovoid, can be employed if desired. The internal diameter of the tubular body 105 is normally in the range of 5 to 20 mm, although other sizes such as larger oval shapes may be employed, if desired. Flexible cannula 100, defines an elongate lumen 110 having openings at both ends of the tubular body 105, defining a proximal opening 102 and distal opening 104. Tubular body 105 is generally flexible, as shown in FIG. 1A, so that it is may conform to the tissue it is inserted through.

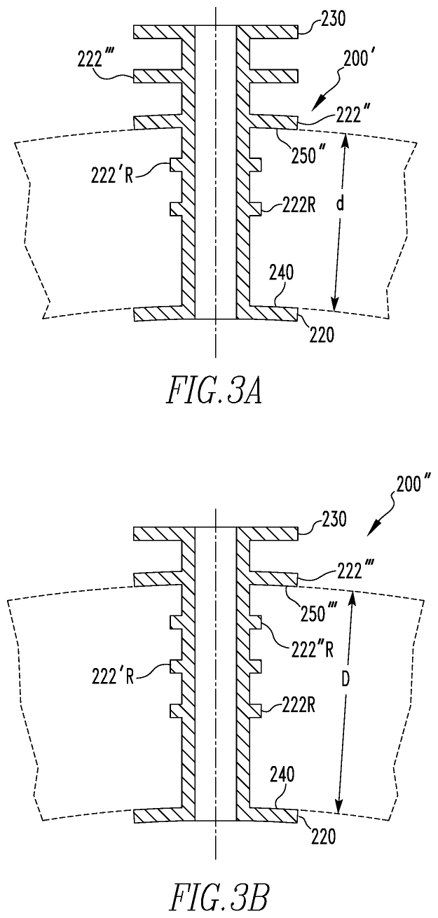

Elongate lumen 110 is generally sized to provide a conduit and allow instrumentation there through into the patient cavity. The plurality of radially extending flanges (120, 122, 122′ . . . and 130) are configured to engage a tissue surface and aid in stabilization of the cannula 100, including a distal flexible flange 120, at a distal terminus of the tubular body 105, a proximal flexible flange 130 at a proximal terminus of the tubular body 105 and a plurality of intermediate flexible flanges (122, 122′, 122″, 122′″ . . . ), extending radially outward from the tubular body outer circumferential surface. All of the plurality of flanges may be similar to each other as shown, in that they may have the substantially equivalent outer diameter (OD) as each other, typical OD being in the range of 6 mm-40 mm, and more preferably in the range 20-35 mm. The proximal flanges (130 and 122, 122′, 122′″ . . . ) are sized and configured so as to engage an outer surface of the tissue, external to a patient cavity and provide port stabilization. The cavity external surface being naturally curved and malleable, but sufficiently planar to provide an approximately perpendicular surface of tissue, perpendicular to the direction of cannula insertion so as to engage with a proximal flange and aid in port stabilization. The flange outer diameters therefore define a radial surface (160, 150, 150′, 150″ . . . ) configured to at least partially engage with the outer tissue surface. Along similar lines, the distal flange OD is configured to as to engage an inner surface of a patient cavity, the inner cavity surface being somewhat curved and malleable by nature, but generally orthogonal to the cannula insertion direction and thereby the tubular body 105, thereby allowing the distal flange radial surface 140 to engage the inner surface and stabilize the flexible port 100 during the procedure, wherein the outer diameter of the distal flange 120 will define a radial surface 140, configured to engage a tissue surface, shown in FIGS. 3A and 3B. Generally, all flanges extend at an approximately perpendicular angle from the tubular body longitudinal axis, however in alternative embodiments the flexible flanges 120 may be disposed at a variety of non-perpendicular angles to the longitudinal axis, to improve insertion or stability of the cannula 100. Each flange (120, 122, 122′ . . . and 130) may also have the same thickness to each other, said thickness being approximately constant or uniform throughout. In alternative embodiments, each flange may radially taper to a thinner outer edge for example, and the more proximal flanges (130 and 122, 122′, 122″ . . . ), may have a differing tapering configuration than the distal flange 120. For example the more proximal flanges (130 and 122, 122′, 122″ . . . ), may define a unilateral taper on a proximal side only (not shown here), having an increased thickness at the intersection with the tubular body while the distal radial surface (150, 160) remains perpendicular to the tubular body 105, the unilateral taper envisioned to inhibit proximal flexing and improve cannula stabilization.

Along similar lines, the distal flange 120 may taper so as to have an increased thickness adjacent the tubular body 105 relative to the outer circumferential portion (not shown here), the taper being unilateral in that it increases on a distal side of the distal flange 120 while the proximal radial surface140 remains perpendicular to the tubular body 105. The cannula 100 may be made from a variety of flexible materials, such as elastomers, polymers and nitinol and while a plurality of materials may be used, the inventors preferably envision the entire flexible cannula to be molded as a single material component. Each flange (120, 130 and 122, 122′, 122″ and 122′″) is axially spaced away from each other and may be equally spaced along the length of the tubular body 105. As shown in FIG. 1C, a cross section of FIG. 1B, it is preferable that distance L between the distal flexible flange 120 and the next adjacent flange 122 be greater than all subsequent flange spacing, corresponding with minimum target tissue depths. Distances l₁, l₂ and l₃ may be substantially similar in distance to each other. In alternative embodiments, l₂ may be less than l₁ and l₃ may be less than l₂ and so on, so as to adjust in incrementally finer gradations along the cannula length for varying tissue depths.

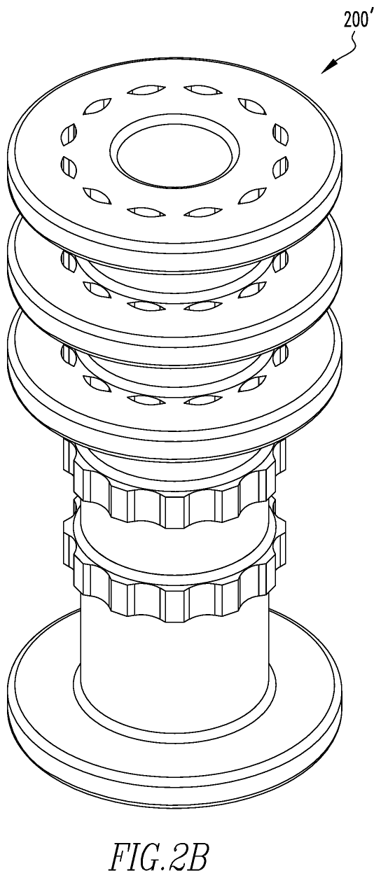

FIGS. 2A and 2B show a further embodiment of a flexible cannula 200 similar to that shown in FIGS. 1A-1C, with the addition of rows of perforations 232 extending circumferentially around at least one flange. Perforations 232 are configured to aid removal of a corresponding flange; so as to aid insertion of the cannula 200 through tissue should the tissue be thicker for example than the distance L, then flange 222 may be removed only, and should for example the tissue thickness be greater than the sum of L and l₁, then flanges 222 and 222′ may be removed. Perforations 232 may extend circumferentially around each flange, radially spaced away from the flange outer circumferential perimeter and also radially spaced from outer surface of the tubular body 205, so as to ensure that the tubular body outer surface is not torn and protect the tubular body inner lumen 210 during flange removal. Perforations 232 may extend through the entire thickness of each corresponding flange and are shaped so as to direct tearing around the flange and not towards the tubular body outer surface 205. As shown, each perforation may taper at each end, in a teardrop or reverse ovate shape so as to create points of stress concentration and influence the direction of tearing between each successive perforation. As shown all proximally disposed flanges 222, 222′, 222″, 222′″ and 230 may have perforations 232. FIG. 2B shows a flexible cannula 200 before and after some of the flanges have been removed (200 and 200′ respectively).

In practice, as illustrated in FIGS. 3A and 3B, at the beginning of a surgical procedure, a tissue depth may have a thickness or depth “d”, corresponding with a distance between distal flange 220 and 222″. Flange 222 and 222′ may therefore be removed prior to insertion of the flexible cannula 200′, leaving a small portion of each flange remaining (labeled 222R and 222′R). The cannula 200′ may be inserted through an incision in the tissue, so as to engage radial surface 240 and 250″ with inner and outer surfaces of tissue defining the tissue depth. Some period of time later during the procedure, the tissue thickness may have changed to a second thickness, “D”, shown in FIG. 3B, that is greater than thickness ‘d”, which often occurs due to patient swelling and/or extravasation. The cannula 200′ may be removed, or left in situ while flange 222″ may be removed, (shown as 222″R in FIG. 3B) leaving the flange 222′″ and 230 remaining to provide stabilization, using flange radial surfaces 240 and 250′.

In a further configuration (not shown) the cannula 200 may be configured so that a proximal portion of the entire tubular body including a flange may be removable. The inventors envisage a ring of perforations extending around and through a portion of the tubular body outer surface, configured to ease removal of a portion of the tubular body (205) as well as any flange associated with that portion. These perforations may be proximally spaced from any fluid valves located within the tubular body lumen that are operable to contain the fluid within a patient cavity. In alternative methods, the cannula tubular body 105 or 205 may be thin enough, or configured so as that it is easy cut with a scalpel or scissors so as to shorten the cannula (100 or 200).

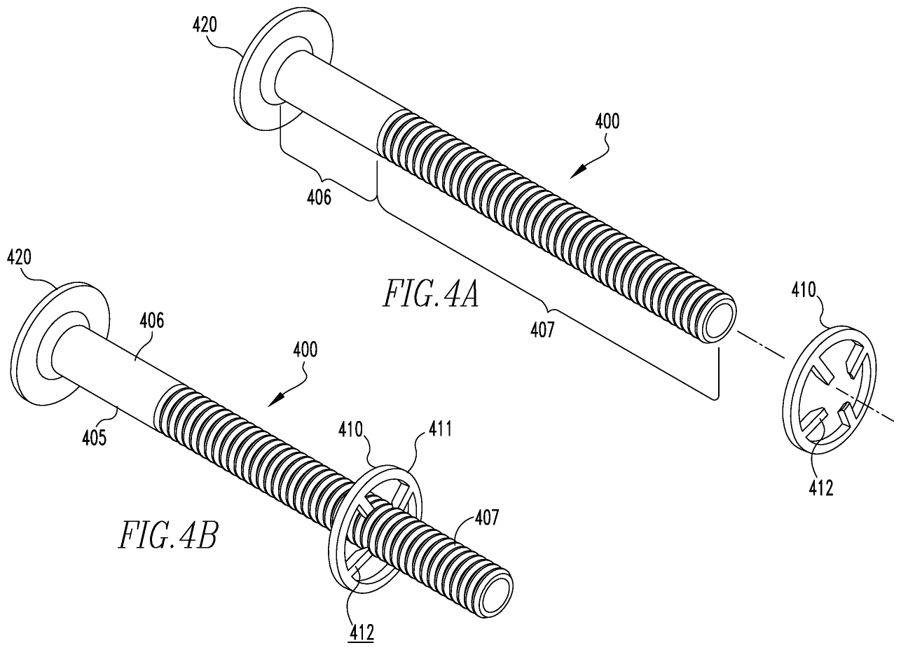

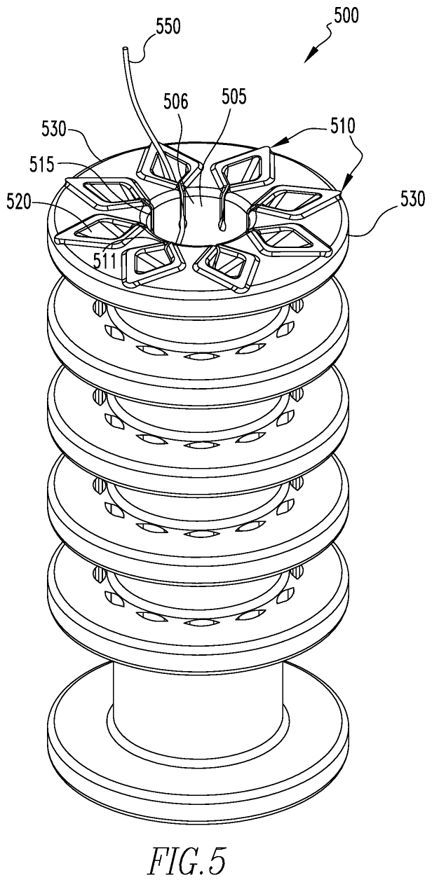

In an alternative embodiment shown in FIGS. 4A and 4B, a flexible cannula 400 may include an adjustable flange 410, adjustable and engageable with the tubular body 405 via a pawl. Similar to previously described flexible cannulas (100, 200), cannula 400 may include a flexible tubular body 405, with a fixed yet conformable distal flange 420. Tubular body 405 may include a distal smooth portion 406, smooth so as to easily insertion through tissue, and a ratchet portion 407 configured to engage a pawl of flange 410 and fix position of flange 410. Adjustable flange 410 may comprise a more rigid material than tubular body 405, through either the inclusion of a stiffer material, and/or though geometry adjustments such as increased thickness, ribs or struts, so as to more securely engage ratcheted portion 407. This embodiment may provide for more finely tuning the location of the flange 410 along the length of the tubular body 405 corresponding with changing tissue thicknesses, and easier adjustment accordingly. Adjustable flange 410 is shown having an outer ring 411, circular in shape and concentrically located relative to the tubular body 405. Four radially extending legs 412 extend from the outer ring, circumferentially spaced equally around the flange 410, the legs 412 configured to selectively engage a portion of the ratcheted tubular body 407 and temporarily fix the flange 410 location. Legs 412 are configured to be sufficiently resilient to resist accidental movement of the flange 410, but also sufficiently flexible to easily allow movement of the flange 410 when required. Such an embodiment may be used in a similar method as described in FIGS. 3A and 3B, and as the tissue becomes thicker, the clinician may observe undue stress on the proximal flange 410 and withdraw the flange proximally. In alternative embodiments, the adjustable flange may move more autonomously, as the legs 412 could be configured to selectively flex upon a given force from the swelling tissue, and could automatically move accordingly. FIG. 5A shows a further embodiment of a flexible cannula 500 with suture docking stations 510 including slits 515 and apertures 520, configured to manage a plurality of sutures or filaments that may extend from inside a body cavity and through the flexible cannula 500. It is also envisioned that a proximal flange with docking stations could be added to the embodiment with an adjustable pawl. As shown in FIG. 5A, a flexible cannula, similar to those described previously (100 and 200) may include these docking stations 510 adjacent to or through a portion of proximal flange 530. Procedures such as rotator cuff repair and superior capsular reconstruction frequently require management of a plurality of sutures through a cannula, which can become tangled, time consuming to manage or difficult to distinguish from each other. Multiple docking locations 510, circumferentially spaced away from each other are therefore envisioned, configured so that sutures 550 (only one shown) can be individually positioned and retained. The intent is that each suture requires some amount of intentional force to remove them from the corresponding slit 515. Eight docking stations 510 are shown, although any number from 1-20 would be plausible. The docking stations 510 include a slotted or slit portion 515 that may extend distally along a portion of the tubular body lumen 505 up to and including the inner lumen surface 506 and may extend radially through a portion of the wall of the tubular body 505 up and including a portion of the proximal flange 530. The combination of friction and elastic properties of the elastomeric flexible cannula 500 with the size of each slit 510 is configured to selectively retain the suture, while the slit is in a relaxed position. Additionally, the slit is configured so as to readily flex and release the length of suture upon tension from a suture end in a first direction. In addition, the slit is configured to retain the suture, but allow the suture to slide therethrough without releasing the length of suture, upon tension is a second direction, different that the first direction. Generally the first direction is radially inward relative to the tubular body 505, and the second direction is either radially away from the longitudinal axis or along the longitudinal axis of the tubular body 505. Extending radially from each slit is a corresponding aperture, shown as being approximately diamond in shape. Generally it was found that an aperture, larger in width than the slit width allowed for a better suture retention, creating a pinch point 511 on each slit, rather than an elongate uniform width channel for example that may hinder sliding of the suture length. Aperture 520 may extend all the way to an outer circumferential surface of the proximal flange 530.

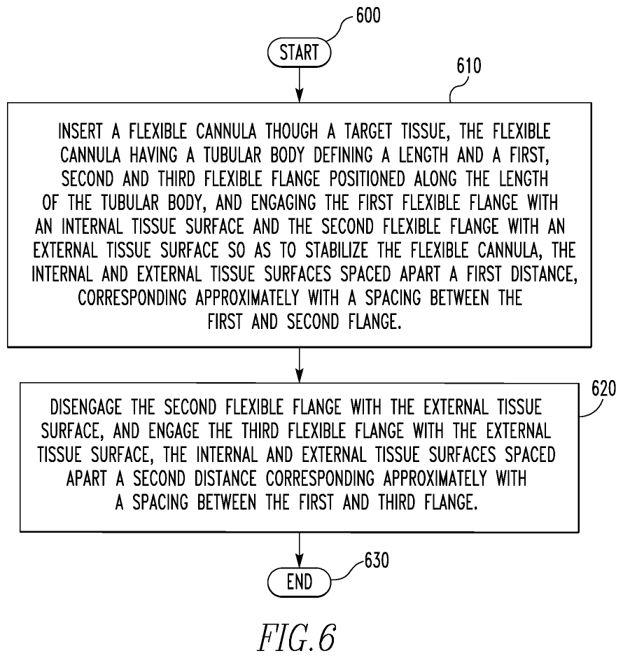

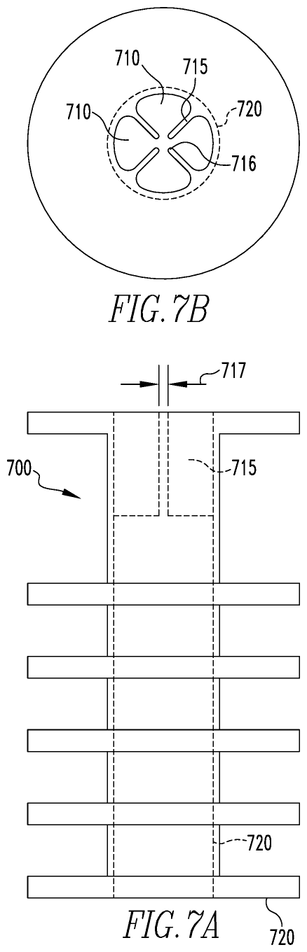

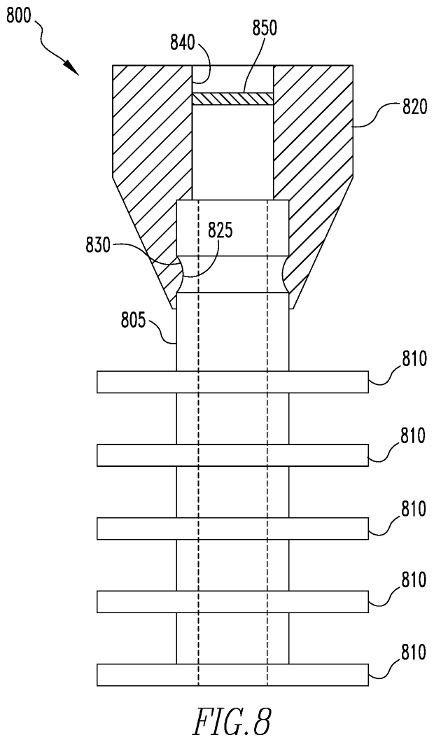

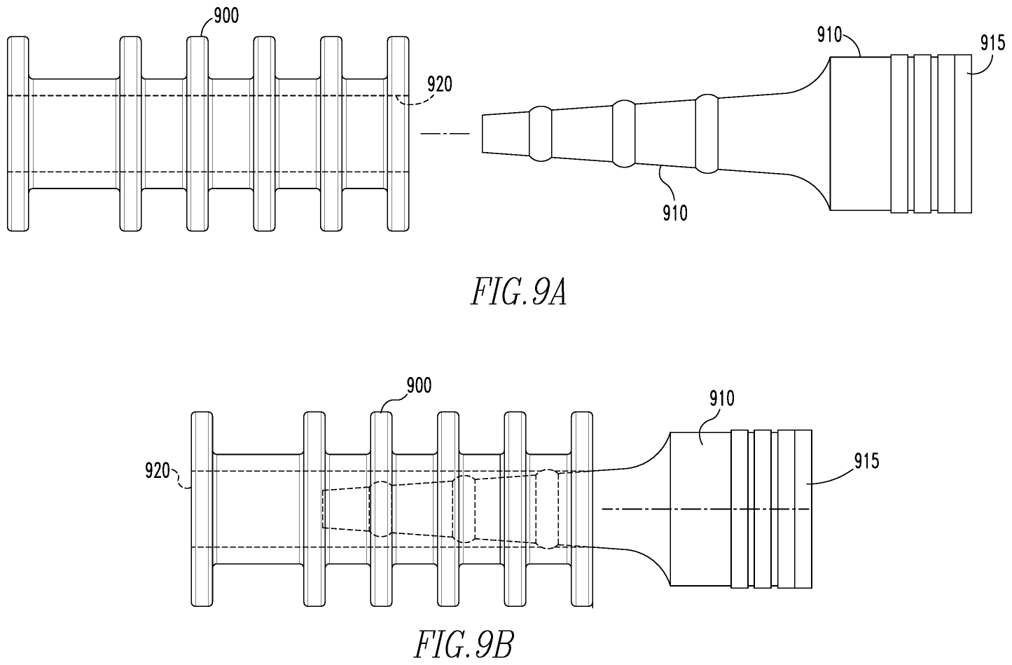

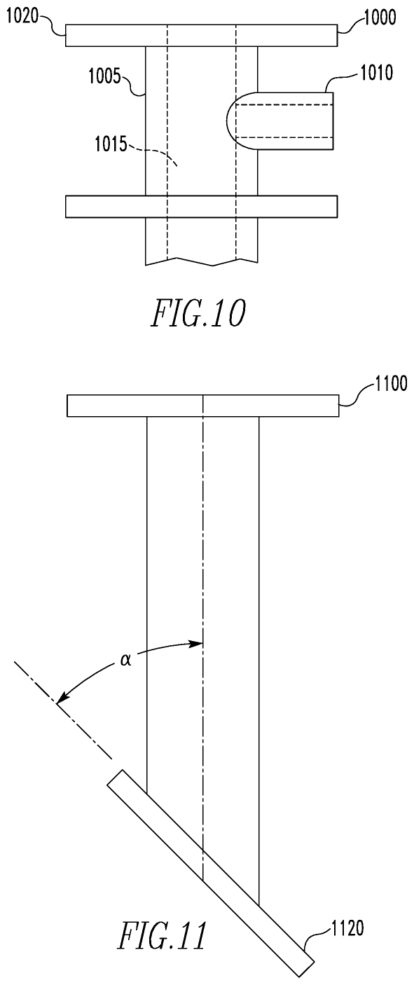

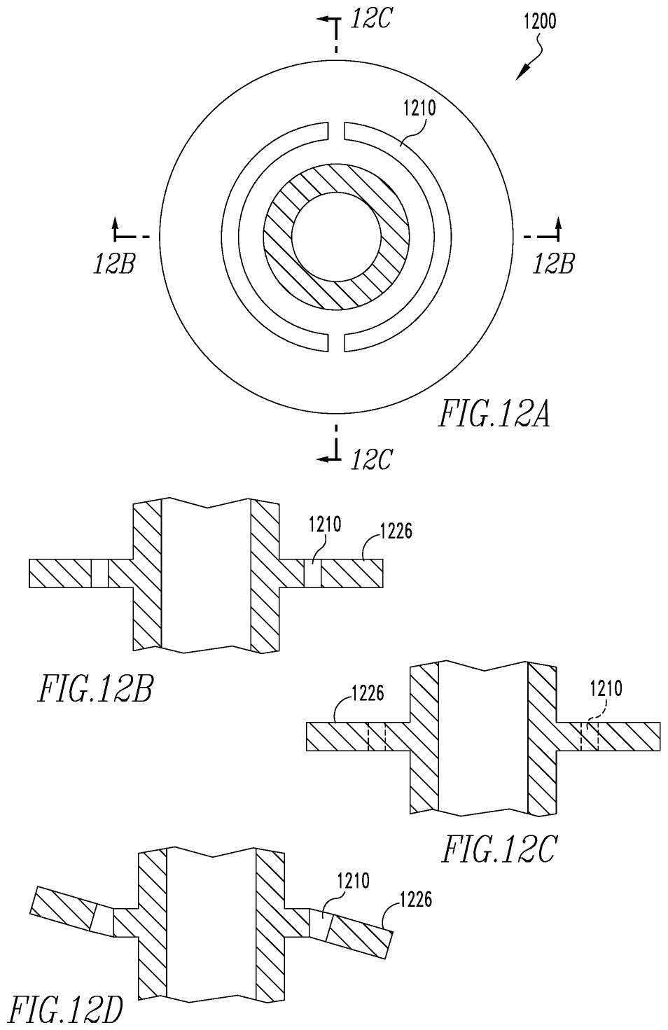

The steps of providing access to a joint is represented in FIG. 6, including beginning 600 and inserting a flexible cannula though an incision in a target tissue, the flexible cannula having a tubular body defining a length and a first, second and third flexible flange positioned along the length of the tubular body, each flange extending radially from the tubular body, 610; and engaging the first flexible flange with an internal tissue surface of a patient cavity, and the second flexible flange with an external tissue surface, so as to define a first stabilizing configuration. The internal and external tissue surfaces are spaced apart a first distance, defining a first tissue depth and the location of the second flange relative to the first flange corresponds approximately with the first tissue depth. For example some compression of the tissue depth may be acceptable by the cannula so as to improve engagement of the flange radial surfaces with the tissue surfaces and aid cannula stabilization, and so the location of the second flange may be a little closer to the first flange than the first tissue depth, for example 0-5 mm. The cannula may include a fourth and fifth flexible flange located along the tubular body and the steps may include removing at least one of the flexible flanges from the flexible cannula, the removing aided by an annular line of perforations disposed through the corresponding flange, and wherein the removal of said flange may be selected based on the distance of this flange from the first flange being less than the first tissue depth, so that this removed flange is between the first and second flange. The steps may also include extending at least one length of suture along the length of the tubular body and retaining the length of suture within a docking station disposed through a proximal end of the tubular body 620. The steps may include disengaging the second flexible flange with the outer tissue surface, due to changing of the tissue depth to a second tissue depth different than the first tissue depth 630. This second tissue depth may be greater than the first due to tissue swelling. Disengaging may be by removal of the second flexible flange from the cannula, by tearing along a row of circumferential perforations. Disengaging may alternatively comprise deforming the second flexible flange and pushing it into the tissue incision. The steps may include engaging the third flexible flange of the flexible cannula with the tissue outer surface, and this may be achieved while the first flexible flange remains stationary, the second tissue depth corresponding approximately with or slightly larger than the distance between the first and third flexible flange.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A flexible cannula for inserting through a target tissue comprising:
a tubular body having a length, a distal flexible flange, a proximal flexible flange, and at least one intermediate flexible flanges all fixedly positioned on the length of the tubular body between the distal and the proximal flanges; wherein the distal flexible flange, proximal flexible flange, and one or more intermediate flexible flanges all define an outer diameter and a radial surface respectively, wherein the distal flexible flange radial surface is configured to engage a first tissue surfaces of the target tissue and wherein the proximal and intermediate flexible flange radial surfaces are configured to selectively engage a second tissue surface opposite the first tissue surface so as to stabilize the flexible cannula.

2. The flexible cannula of claim 1 wherein the flexible flanges have a relaxed position that extends perpendicular to the tubular body elongate axis.

3. The flexible cannula of claim 1 wherein the one or more intermediate flexible flanges comprise a plurality of intermediate flexible flanges equally spaced from each other along the length of the tubular body.

4. The flexible cannula of claim 1 wherein one or more intermediate flexible flanges comprises a distal intermediate flange and a proximal intermediate flange, and wherein the distal intermediate flange is spaced further from the distal flexible flange than the proximal intermediate flange.

5. The flexible cannula of claim 1 wherein the tubular body has a membrane positioned across an inner diameter, and wherein the membrane has a slit to allow passage of instruments therethrough.

6. The flexible cannula of claim 1 wherein the one or more intermediate flanges comprise perforations configured to ease selective removal of the intermediate flanges.

7. The flexible cannula of claim 6 wherein the perforations define a circumferential row adjacent to but not intersecting the tubular body.

8. The flexible cannula of claim 1, the tubular body having a ring of perforations positioned around an outer circumference of the tubular body to allow for easy removal of a portion of the length of the cannula.

9. The flexible cannula of claim 1, wherein the tubular body proximal end comprises at least one, slit for selectively retaining a length of suture therein, the at least one slit extending radially from an inner surface of the tubular body and through a portion of the proximal flange.

10. A flexible cannula comprising:
a tubular body defining a length, a proximal and distal end, a distal flexible flange extending radially from the tubular body distal end, a proximal flange flexible extending radially from the tubular body proximal end and one or more intermediate flexible flanges positioned along the length of the tubular body, between the distal and the proximal flanges; wherein the tubular body proximal end comprises at least one slit extending radially from, an inner surface of the tubular body and through a portion of the proximal flange; the at least one slit configured to selectively retain a length of suture.

11. The flexible cannula of claim 10 wherein the distal flange, the proximal flange and the one or more intermediate flanges all have an outer diameter that is the equivalent.

12. The flexible cannula of claim 10, wherein the cannula including all of the flanges are molded of a single elastomeric component.

13. The flexible cannula of claim 10, wherein the tubular body defines an inner lumen having an inner diameter, the inner lumen comprising a membrane positioned across the inner diameter, and wherein the membrane and has a slit to allow passage of instruments.

14. The flexible cannula of claim 10, wherein the one or more intermediate flanges have an annular row of perforations adjacent the tubular body, configured for ease of flange removal.

15. The flexible cannula of claim 10, wherein the tubular body has one or more rings of perforations positioned around the tubular body to allow for easy removal of a portion of the cannula.

16. A method of providing access to a joint comprising;
inserting a flexible cannula though a target tissue, the flexible cannula having a tubular body defining a length and a first, second and third flexible flange fixedly positioned along the length of the tubular body;
engaging the first flexible flange with an internal tissue surface and engaging the second flexible flange with an external tissue surface so as to stabilize the flexible cannula, the internal and external tissue surfaces spaced apart a first distance, defining a first tissue depth corresponding approximately with a distance between the first and second flange along the length of the tubular body.

17. The method of claim 16, wherein the flexible cannula further comprises a fourth flexible flange, disposed between the first and second flexible flange, and prior to inserting the flexible cannula, removing the fourth flexible flange from the flexible cannula.

18. The method of claim 16 wherein a least some of the flexible flanges comprise an annular line of perforations configured to aid in selectively removing the flexible flanges from the flexible cannula.

19. The method of 16 further comprising extending at least one length of suture along the length of the tubular body and retaining the length of suture within a docking station comprising a slit disposed through a proximal end of the tubular body.

20. The method of claim 16 further comprising disengaging the second flexible flange with the external tissue surface, and engaging the third flexible flange with the external tissue surface, the internal and external tissue surfaces spaced apart a second distance, defining a second tissue depth corresponding approximately with a distance between the first and third flange.
